(19) United States
(12) Patent Application Publication
(10) Pub. No.: US 2025/0114230 A1
(43) Pub. Date: Apr. 10, 2025

(54) AN OSTOMY DRAINAGE SYSTEM

(71) Applicant: SALTS HEALTHCARE LIMITED, West Midlands (GB)

(72) Inventors: Lee Howard, West Midlands (GB); Kieran Williams, West Midlands (GB); Lee Smith, West Midlands (GB)

(21) Appl. No.: 18/836,566

(22) PCT Filed: Feb. 22, 2023
(86) PCT No.: PCT/GB2023/050392

§ 371 Date: Aug. 7, 2024

(30) Foreign Application Priority Data
Feb. 22, 2022 (GB) 2202398.0

(51) Int. Cl. A61F 5/44; A61F 5/445

(52) U.S. Cl. CPC A61F 5/4405; A61F 5/445

(57) ABSTRACT

An ostomy drainage system including an ostomy appliance and a drainage conduit, the ostomy appliance including an internal collecting volume defined by a first wall and a second wall connected together; a stoma receiving opening defined in the first wall, and a valve. The valve includes a body connected to the first and second walls, an inlet and an outlet, connected by a flow path and fluidly connected to the internal collecting volume. The drainage conduit includes a connector device which is connectable to the valve. The connector device has a connector body providing a passage, and an inlet formation which widens radially outwardly relative to the connector body, such that when the connector device is connected to the valve, the connector body extends from the outlet to the inlet and the inlet formation resists removal from the valve.

### CROSS-REFERENCE TO RELATED APPLICATIONS

This application is a national phase filing under 35 § U.S.C. 371 of International Application No. PCT/GB2023/050392, filed on Feb. 22, 2023, which claims priority to British Patent Application No. 2202398.0, filed on Feb. 22, 2022, the entire contents of each of which are incorporated herein by reference.

### BACKGROUND OF THE INVENTION

Urostomy appliances are well known in the field. They are typically attached to a user via an adhesive member that extends around the user's stoma with adhesive and provide a collecting volume to collect waste (mostly liquid waste) exiting the stoma. A mechanism for draining the collecting volume is often provided—typically, these are in the form of a tap, so that a user can empty waste that is collected in the collecting volume without having to remove and dispose of the entire appliance.

At times, a user may want to extend the time between emptying their appliance, for example, when they are sleeping. Typically, a larger capacity bag is used to contain waste while the user is sleeping. In this scenario, the outlet valve is connected to the larger capacity bag by a tube, so that the bag fills with waste.

Embodiments of the present invention seek to alleviate one or more problems associated with the prior art.

### SUMMARY

According to a first aspect of the invention we provide an ostomy drainage system including an ostomy appliance and a drainage conduit, the ostomy appliance including:
an internal collecting volume defined by a first wall and a second wall connected together about a periphery;
a stoma receiving opening defined in the first wall, and
a valve including:
   a body connected to the first and second walls,
   an inlet and an outlet, connected by a flow path and fluidly connected to the internal collecting volume; and
the drainage conduit including a connector device which is connectable to the valve, wherein the connector device has a connector body providing a passage, and an inlet formation which widens radially outwardly relative to the connector body, such that when the connector device is connected to the valve, the connector body extends from the outlet to the inlet and the inlet formation resists removal from the valve.

The inlet formation may form part of a mucus guard member. The connector device may be connected to the valve. The connector body may extend from the outlet to the inlet. The mucus guard member may be located at the inlet and may provide a cage around the inlet to inhibit the ingress of mucus. The mucus guard member may include a generally upstanding wall which, in use, projects away from the inlet of the valve. The wall may project from the connector body in an annular shape. The mucus guard member may include a plurality of slots or channels, and optionally the slots/channels may be spaced around the inlet. The mucus guard member may have four slots. The first and second slots may be separated by a bar of the cage and form a first pair and the third and fourth slots may be separated by a second bar of the cage and form a second pair. The first pair may be on an opposing side of the mucus guard member to the second pair. The mucus guard member may form a frustoconical shape.

The shape of the mucus guard member may be wider at a base and tapers to an end. The base may be between 0.8 and 1.2 cm wide and preferably around 0.95 cm wide. The end of the mucus guard member may be between 0.7 and 0.95 cm wide.

The connector body may include a shoulder formation. The shoulder formation may be positioned adjacent the outlet of the valve when in use. The connector body and/or the mucus guard member may be formed from a rigid material and optionally a rigid plastics material. The connector body and the mucus guard member may be formed from the same material.

The mucus guard member may be between 0.8 and 1.2 cm long and preferably around 1 cm long. The connector body may be between 0.9 and 1.3 cm long and preferably around 1.1 cm long. A ratio of the length of the connector body to the length of the mucus guard member may be between 1:1 and 1.5:1 and may be preferably around 1.1:1.

The connector device may provide an anchor that holds the connector device connected to the valve and a mucus guard member that prevents/reduces how much mucus travels into the passage.

### BRIEF DESCRIPTION OF THE FIGURES

In order that the present disclosure may be more readily understood, preferable embodiments thereof will now be described, by way of example only, with reference to the accompanying drawings, in which:

FIG. 1 is a schematic view of an ostomy drainage system;

FIG. 2 is a schematic side cross-sectional view of an ostomy appliance;

FIGS. 3A and 3B show different views of part of a drainage conduit;

FIG. 4 is a perspective view of part of the drainage conduit and a valve of an ostomy appliance; and

FIG. 5 shows part of the drainage conduit connected to the valve of the ostomy appliance.

### DETAILED DESCRIPTION OF THE DISCLOSURE

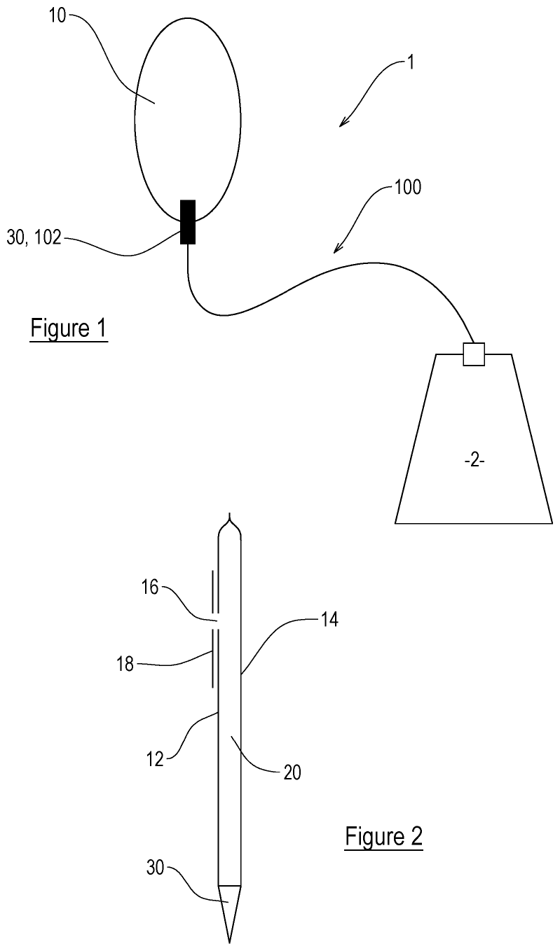

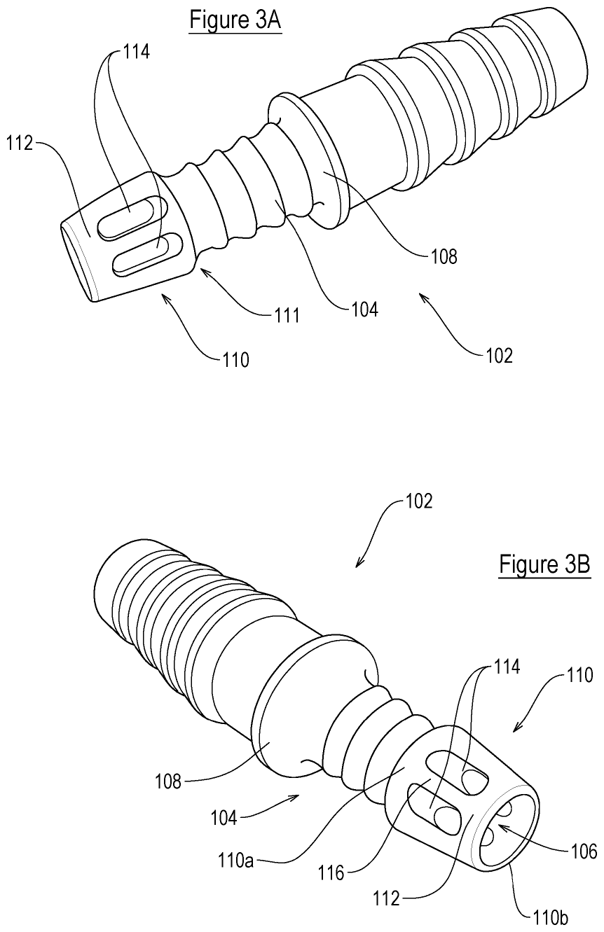

Referring to the FIG. 1, an ostomy drainage system 1 is illustrated. The ostomy drainage system 1 includes an ostomy appliance 10 and a drainage conduit 100.

Referring to FIG. 2, an ostomy appliance 10 (or appliance 10) is illustrated. The ostomy appliance 10 includes a first wall 12 and a second wall 14. The first and second wall 12, 14 are connected together about a periphery to define an internal collecting volume 20. In the illustrated example, the first wall 12 is on a “body” side of the appliance 10 (i.e. it is closer to the body than the internal collecting volume when the appliance 10 is in use) and the second wall 14 is on a “non-body” side of the appliance 10 (i.e. it is further/furthest from the body than the internal collecting volume when the appliance 10 is in use).

A stoma receiving opening 16 is defined in the first wall 12. The stoma receiving opening 16 is in fluid communication with the internal collecting volume such that in use, waste entering the appliance via the stoma receiving opening flows into the collecting volume. In some embodiments, an adhesive member 18 is attached to the first wall 12 and extends around the stoma receiving opening 16. The adhesive member 18 permits a user of the appliance 10 to attach the appliance 10 to their abdomen around their stoma.

A valve 30 is provided to allow waste to be emptied from the internal collecting volume 20. The valve 30 includes a body 32, an inlet 40 and an outlet 42. The body 32 is connected to the first and second walls 12, 14. The inlet 40 and the outlet 42 are connected by a flow path and are fluidly connected to the internal collecting volume 20.

The valve 30 includes a closure member (not shown) which is used to close the valve 30. The closure member is inserted into the outlet 42 and blocks the flow path out of the appliance 10. When a user wishes to open the valve 30 (i.e. to empty their appliance 10 or to connect a drainage conduit 100 as discussed below), the closure member is removed from the outlet 42 so that the flow path is open and waste can flow from the internal collecting volume 20 through the valve and out of the appliance 10.

As mentioned above, the drainage system 1 further includes a drainage conduit 100. The drainage conduit 100 includes a connector device 102 which is connectable to the valve 10. The connector device 102 has a connector body 104 and an inlet formation 111. In some embodiments, the inlet formation 111 forms part of a mucus guard member 110.

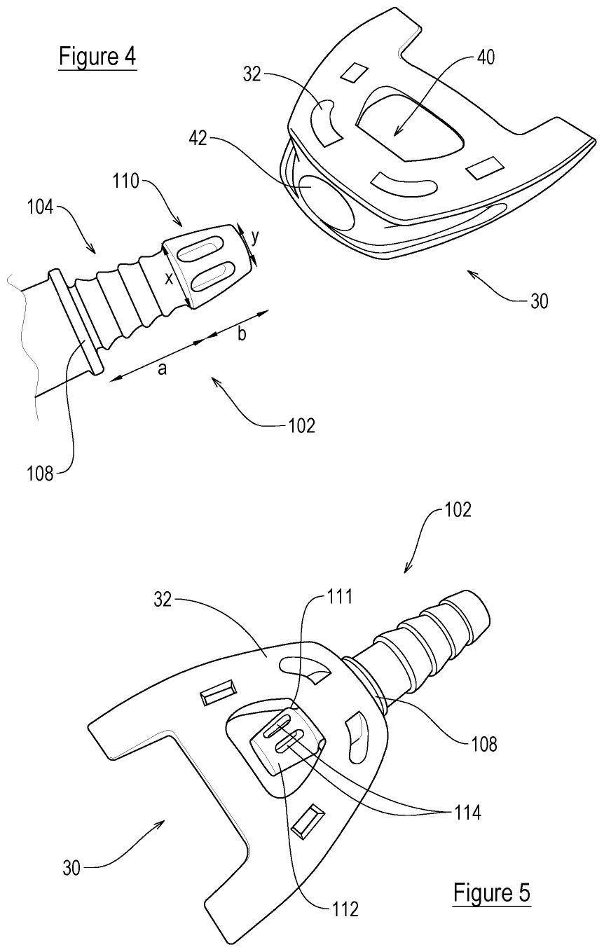

The connector body 104 has a central bore that provides a passage 106. An exterior surface of the connector body 104 includes multiple tapered surfaces, each of which extends axially along the connector body 104 and is adjacent the next tapered surface. Thus, the exterior surface of the connector body 104 has a series of wedge shaped parts that form the surface. In some embodiments, the connector body 104 is between 0.9 and 1.3 cm long and preferably around 1.1 cm long (illustrated by “a” in FIG. 4).

In some embodiments, the connector body 104 includes a shoulder formation 108. In other words, the connector body 104 has an outwardly projecting flange that extends around the body 104. The shoulder formation 108 is spaced from the mucus guard member 110 (and, in the illustrated example, the tapered exterior surface of the connector body 104 is located adjacent the shoulder formation 108 and between the shoulder formation 108 and the mucus guard member 110).

The inlet formation 111 widens radially outwardly relative to the connector body 104. In other words, the inlet formation 111 provides a conical or outwardly tapering part that extends outwardly of the connector body 104. The inlet formation 111 may form part of the mucus guard member 110 described below or it may be provided alone.

The mucus guard member 110 provides a cage around an end of the connector body 104 (i.e. a formation that provides multiple “bars” or projections away from the connector body 104 that catches/slows mucus flowing towards the passage 106). The mucus guard member 110 provides a flow path into the connector body 104/conduit 100 for liquid waste but inhibits the progress of more viscous material into the passage 106. In other words, the end of the drainage conduit 100 has a sieve-like/filter formation around it to prevent (or at least inhibit and slow down) the ingress of mucus into the conduit 100.

In some embodiments, the mucus guard member 110 forms a frustoconical shape. In other words, a base 110a of the mucus guard member 110 is wider than an end 110b. The mucus guard member 110 tapers gradually from the base 110a to the end 110b. In the present example, the base 11a is between 0.8 and 1.2 cm wide and preferably around 0.95 cm wide (see line “x” on FIG. 4). Further, the end 110b of the mucus guard member 110 is between 0.5 and 0.9 cm wide and preferably around 0.7 cm wide (see line “y” on FIG. 4). It should be appreciated that the mucus guard member 110 may not form a frustoconical shape—in which case both the base and the end may be around 0.95 cm.

In some embodiments, the mucus guard member 110 is between 0.8 and 1.2 cm long and preferably around 1 cm long (see line “b” in FIG. 4). Thus, a ratio of the length of the connector body (line “a”) to the length of the mucus guard member (line “b”) is between 1:1 and 1.5:1. In the present example, the total length of the connector device 102 is around 2.1 cm, where the connector body 104 is around 1.1 cm and the mucus guard member 110 is around 1.0 cm (thus, the ratio of body 104 length to member 110 length is around 1.1:1).

It should be appreciated that the mucus guard member 110 could have a longer length and provide more apertures to form the sieve/filter for the mucus. For example, it could be up to 30 to 50 mm. In such an example, the material may be softer (e.g. a TPE) and the length would be chosen to avoid interfering with a non-return valve within the ostomy appliance 10 itself (the non-return valve is formed of layers of film that ensures waste entering the appliance 10 moves away from the stoma area and cannot return).

In some embodiments, the mucus guard member 110 includes a generally upstanding wall 112. The upstanding wall 112 projects from the connector body 104 in an annular shape. In the present example, a circular opening is provided in the end of the mucus guard member 110. However, this need not be the case and one or more additional structural members may be provided across the passage to provide further prevention of mucus into the passage 106.

The mucus guard member 110 includes a plurality of slots 114 (and, preferably, has four slots 114) formed in the wall 112. In this context a slot is considered to be substantially elongated oval shape or a stadium shape and extends through the material of the mucus guard member 110 (i.e. through the wall 112) to provide a small opening. In some embodiments, each opening is around 6 mm long and 1.9 mm wide.

It should be appreciated, that each slot could be formed as a recess instead. In other words, a channel would be formed extending from the end 110b of the mucus guard member 110 towards the base 110a.

In the illustrated example, first and second slots 114 are formed adjacent each other with a structure member 116 provided in between. Further, the third and fourth slots 114 are also formed adjacent each other with a structural member 116 provided between them. In other words, the first and second slots 114 form a first pair and the third and fourth slots 114 form a second pair.

In the present example, the first pair is positioned on a first side of the mucus guard member 110 and the second pair is positioned on a second, opposing, side of the mucus guard member 110. In other words, the cage of the mucus guard member 110 mentioned above is formed from the wall 112 and the slots 114 distributed around the wall 112 (they may form an evenly distributed annular arrangement). The structural members 116 form the “bars” of the cage.

In some embodiments, each of the slots 114 is positioned the same distance from a plane that extends transversely/perpendicularly through the connector body 104. As such they lie the same distance from the inlet 40 of the valve 30 during use.

In some embodiments, the connector body 104 and/or the mucus guard member 110 are formed from a rigid material. The rigid material may preferably be a rigid plastics material.

The features of the drainage conduit 100 will now be discussed with reference to where they are located when in use (i.e. when the connector device 102 is connected to the valve 30). This is shown in FIG. 5.

The connector device 102 is inserted into the outlet 42 of the valve 30 and extends through the inlet 40 into the interior of the appliance 10. The connector body 104 extends from the outlet 42 to the inlet 40. Thus, the multiple tapered surfaces discussed above engage with an interior surface of the valve 30 (in the flow path between the inlet 40 and the outlet 42). The shaped exterior surface of the connecter body 104 aids the sealing contact against the valve 30 and stops leakage of waste between the valve 30 and the connector device 102.

The inlet formation 111 flares outwardly from the inlet of the valve 30 (this inlet formation 111 is wider than the inlet 40). Thus, the inlet formation 111 provides an anchor function/resists removal from the valve 30. In other words, when outward force is applied the connector device 102 (i.e. away from the outlet 42 and the appliance 10), the inlet formation 111 inhibits removal of the connector device 102 from the valve 30.

As can be seen in FIG. 5, the shoulder formation 108 is positioned adjacent the outlet 42 of the valve 30. This flanged portion prevents the connector body 104 from being inserted into the valve 30 too far and avoids damage or distortion of the valve body 32.

The mucus guard member 110 (which is at the end of the connector body 104) is located at or adjacent the inlet 40 and provides the case around the inlet 40 to inhibit the ingress of mucus into the passage 106.

In other words, the mucus guard member 110 is positioned over the inlet 40 so that liquid is not prevented from exiting through the inlet 40 but heavier/more viscous material is impeded by the slots 114 in the side of the mucus guard member 110.

The wall 116 projects away from the inlet 40 (and into the appliance 10). As discussed above, the mucus guard member 110 is only around 1 cm long, so it does not project far into the appliance 10. It is positioned only to impede the flow of mucus out of the appliance 10.

The ostomy drainage system 1 is designed for urostomy or ileostomy use where the waste excreted by a user's stoma is liquid or predominantly liquid (i.e. urine or a urine/mucus mix). The drainage system 1 is primarily for use during the night/when a user does not want to or cannot empty their urostomy appliance. The drainage conduit 100 includes a conduit that extends to a further collecting volume 2 (i.e. in addition to the internal collecting volume 20 itself).

The present invention seeks to prevent mucus build up in the conduit (which can lead to blockages and prevents urine from reaching the further collecting volume 2). This is addressed by providing a mucus guard member 110 that forms a cage around the inlet 40 of the valve 30 (and around the passage 106 of the connector device 102). It is important that the slots 114 and passage 106 are close to the inlet 40, so that waste does not collect near the valve 30 and potentially cause leaking but also so that the mucus is held out of the passage 106 and the liquid can flow more freely.

When used in this specification and claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure.

Although certain example embodiments of the invention have been described, the scope of the appended claims is not intended to be limited solely to these embodiments. The claims are to be construed literally, purposively, and/or to encompass equivalents.

1. An ostomy drainage system including an ostomy appliance and a drainage conduit, the ostomy appliance including:
an internal collecting volume defined by a first wall and a second wall connected together about a periphery;
a stoma receiving opening defined in the first wall, and
a valve including:
a body connected to the first and second walls,
an inlet and an outlet, connected by a flow path and fluidly connected to the internal collecting volume; and
the drainage conduit including a connector device which is connectable to the valve, wherein the connector device has a connector body providing a passage, and an inlet formation which widens radially outwardly relative to the connector body, such that when the connector device is connected to the valve, the connector body extends from the outlet to the inlet and the inlet formation resists removal from the valve.

2. An ostomy drainage system according to claim 1 wherein the inlet formation forms part of a mucus guard member.

3. An ostomy drainage system according to claim 2 wherein, when the connector device is connected to the valve, the connector body extends from the outlet to the inlet and the mucus guard member is located at the inlet and provides a cage around the inlet to inhibit the ingress of mucus.

4. An ostomy drainage system according to claim 2 wherein the mucus guard member includes a generally upstanding wall which, in use, projects away from the inlet of the valve.

5. An ostomy drainage system according to claim 4 wherein the wall projects from the connector body in an annular shape.

6. An ostomy drainage system according to claim 2 wherein the mucus guard member includes a plurality of slots or channels, and optionally the slots/channels are spaced around the inlet.

7. An ostomy drainage system according to claim 6 where in the mucus guard member has four slots.

8. An ostomy drainage system according to claim 7 wherein the first and second slots are separated by a bar of the cage and form a first pair and the third and fourth slots are separated by a second bar of the cage and form a second pair.

9. An ostomy drainage system according to claim 8 wherein the first pair is on an opposing side of the mucus guard member to the second pair.

10. An ostomy drainage system according to claim 2 wherein the mucus guard member forms a frustoconical shape which is wider at a base and tapers to an end.

11. An ostomy drainage system according to claim 10 wherein the base is between 0.8 and 1.2 cm wide and preferably around 0.95 cm wide.

12. An ostomy drainage system according to claim 10 wherein the end of the mucus guard member is between 0.7 and 0.95 cm wide.

13. An ostomy drainage system according to claim 1 wherein the connector body includes a shoulder formation that is positioned adjacent the outlet of the valve when in use.

14. An ostomy drainage system according to claim 1 wherein the connector body is formed from a rigid material and optionally a rigid plastics material

15. An ostomy drainage system according to claim 2 wherein the mucus guard member is formed from a rigid material and optionally a rigid plastics material.

16. An ostomy drainage system according to claim 15 wherein the connector body and the mucus guard member is formed from the same material.

17. An ostomy drainage system according to claim 2 wherein the mucus guard member is between 0.8 and 1.2 cm long and preferably around 1 cm long.

18. An ostomy drainage system according to claim 1 wherein the connector body is between 0.9 and 1.3 cm long and preferably around 1.1 cm long.

19. An ostomy drainage system according to claim 2 wherein a ratio of the length of the connector body to the length of the mucus guard member is between 1:1 and 1.5:1 and preferably around 1.1:1.

20. An ostomy drainage system according to claim 1 wherein the connector device provides an anchor that holds the connector device connected to the valve and a mucus guard member that prevents/reduces how much mucus travels into the passage.
